(19) United States
(12) Patent Application Publication
(10) Pub. No.: US 2025/0216700 A1
(43) Pub. Date: Jul. 3, 2025

(54) REACTIVE UV ABSORBERS AND ANTI-BLUE LIGHT EYE DEVICE COMPRISING THE SAME

(71) Applicant: Everlight Chemical Industrial Corporation, Taipei City (TW)

(72) Inventors: Jui-Lin WU, Taoyuan City (TW); Zhen-Jie FAN, Taoyuan City (TW); Chia-Sheng CHIANG, Taoyuan City (TW); Chi-Hsiang YAO, Taoyuan City (TW)

(21) Appl. No.: 19/001,798

(22) Filed: Dec. 26, 2024

(30) Foreign Application Priority Data
Dec. 29, 2023 (TW) 112151529

(51) Int. Cl. G02C 7/04; C07D 249/20; G02B 1/04; G02C 7/10

(52) U.S. Cl. CPC G02C 7/049; C07D 249/20; G02B 1/04; G02C 7/104; G02C 7/108

(57) ABSTRACT

A novel reactive UV absorber and an anti-blue light eye device comprising the same are provided. The novel reactive UV absorber is a compound represented by the following formula (I):

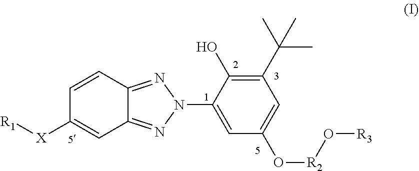

wherein X, R₁, R₂ and R₃ are defined in the specification.

### CROSS-REFERENCE TO RELATED APPLICATIONS

This application claims the benefits of the Taiwan Patent Application Serial Number 112151529, filed on Dec. 29, 2023, the subject matter of which is incorporated herein by reference.

### BACKGROUND OF THE INVENTION

### Field

The present invention relates to a novel reactive UV absorbers and an anti-blue light eye device comprising the same. More particularly, the present invention relates to a benzotriazole UV absorber with a reactive functional group and an anti-blue light eye device comprising the same.

### Description of Related Art

Ultraviolet light absorber (UV absorber) is a kind of light stabilizer, which can absorb ultraviolet rays in sunlight or fluorescent light sources, thereby protecting the material added with a UV absorber from being damaged by ultraviolet rays. Alternatively, when a coating containing a UV absorber is applied to a substrate, it can also protect the substrate from UV damage.

On the other hand, high-energy blue light (380-460 nm) is associated with some eye diseases (such as macular degeneration, cataracts, etc.). However, currently available UV absorbers still have their shortcomings. For example, if the anti-blue light benzotriazole UV absorber does not have reactive functional groups, migration may occur in vinyl polymer materials. In addition, commercially available UV absorbers, such as EV73 and RUVA93, are less effective at absorbing blue light with wavelengths above 400 nm.

Therefore, it is desirable to provide a novel benzotriazole UV absorber to solve the aforesaid shortcomings.

### SUMMARY OF THE INVENTION

An object of the present invention is to provide a novel compound with excellent blue light blocking rate. The novel compound disclosed by the present invention has the structure represented by the following formula (I):

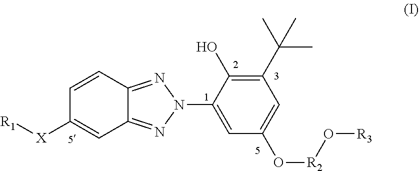

wherein X is —S— or —SO₂—;
R₁ is phenyl or tolyl;
R₂ is linear C₃₋₅ alkylene or a carbamate-containing group; and
R₃ is a C₂₋₅ alkenyl-containing group.

As shown in the above formula (I), the novel compound provided by the present invention is a benzotriazole UV absorber with both the reactive functional group and anti-blue light function. When the novel compound provided by the present invention is applied to an anti-blue light eye device, for example, polymerized with monomers for contact lens to form a film, the average blue light blocking rate of the obtained contact lens film can reach more than 48-65%. Therefore, the novel compound provided by the present invention can be applied to anti-blue light eye devices (such as contact lenses, intraocular lenses, etc.).

In one aspect of the present invention, X is —S— or —SO₂—.

In one aspect of the present invention, R₁ is phenyl or tolyl. Herein, when R₁ is tolyl, X and methyl are in the para position of the phenyl group.

In one aspect of the present invention, R₂ is linear C₃₋₅ alkylene or a carbamate-containing group. In one aspect of the present invention, R₂ may be linear C₃₋₅ alkylene or —R₄—OC(═O)NHR₅—, wherein R₄ may be linear C₃₋₅ alkylene and R₅ may be linear C₁₋₃ alkylene. In another aspect of the present invention, R₂ may be propylene or —R₄—OC(═O)NHR₅—, wherein R₄ may be propylene and R₅ may be ethylene.

In one aspect of the present invention, R₃ is a C₂₋₅ alkenyl-containing group. In one aspect of the present invention, R₃ may be a group containing C₂₋₅ alkenyl and carbonyl. In one aspect of the present invention, R₃ may be —C(═O)—R₆ and R₆ may be C₂₋₅ alkenyl. In another aspect of the present invention, R₃ may be —C(═O)—R₆ and R₆ may be propenyl.

In one specific aspect of the present invention, the compound of formula (I) may be any compound of the following formulas (I-1) to (I-4):

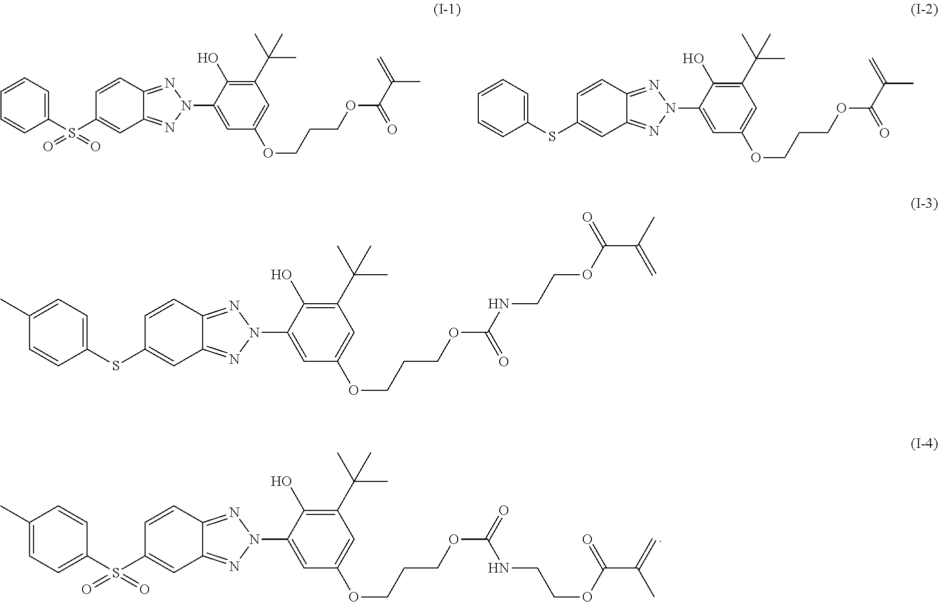

In the present invention, specific examples of “alkyl(ene)” include, but are not limited to, ethyl(ene), propyl(ene), butyl(ene), and pentyl(ene).

In the present invention, the term “alkenyl” refers to a linear or branched hydrocarbon group containing at least one double bond, for example, a linear or branched C₂₋ₛ hydrocarbon group containing at least one double bond. Specific examples thereof include, but are not limited to, ethylene, propylene, and butylene.

In addition, the present invention further provides a composition that is stable against photo-induced degradation, which comprises: (A) a photo-induced degradable organic material; and (B) the aforesaid novel compound of the present invention. Herein, the content of the novel compound of the present invention may range from 0.1 wt % to 30 wt % based on the weight of the organic material.

In one aspect of the present invention, the aforesaid composition is used for forming a coating layer. In particular, the coating layer is formed on a substrate which is sensitive to electromagnetic radiation with a wavelength greater than 380 nm. The material of the substrate is not particularly limited, and can be glass, plastic, polymer, silicone hydrogel, resin, carbon fiber complex material, or a combination thereof.

In addition, the present invention further provides a protection film with anti-blue light effect, which comprises the aforesaid novel compound of the present invention. Herein, the novel compound of the present invention is applied to a substrate for the protection film to form an anti-UV or anti-blue light coating layer.

Furthermore, the present invention further provides an anti-blue light eye device with anti-blue light effect, which comprises the aforesaid novel compound of the present invention. Herein, specific examples of the anti-blue light eye device include, but are not limited to, a contact lens (for example, a soft contact lens or a hard contact lens), an implantable ophthalmic lens or an intraocular lens.

In one aspect of the present invention, the method for manufacturing an anti-blue light eye device may comprise the following steps: mixing the aforesaid novel compound of the present invention with a matrix to form a mixing solution, wherein the content of the aforesaid novel compound of the present invention may be between 0.1 wt % and 10.0 wt %, and the content of the matrix may be between 90.0 wt % and 99.9 wt %. In one aspect of the present invention, the content of the aforesaid novel compound of the present invention may be between 0.1 wt % and 5.0 wt %, and the content of the matrix may be between 95.0 wt % and 99.9 wt %. In another aspect of the present invention, the content of the aforesaid novel compound of the present invention may be between 0.1 wt % and 1.0 wt %, and the content of the matrix may be between 99.0 wt % and 99.9 wt %.

In one aspect of the present invention, the matrix may comprise a hydrophilic substance, a polymerization initiator, a cross-linking agent or a combination thereof. Examples of the hydrophilic substance may comprise, but are not limited to, 2-hydroxyethyl methacrylate (HEMA), methacrylic acid (MAA), acrylic acid (AA), N-vinylpyrrolidone (NVP), N,N-dimethylacrylamide (DMAA), glycidyl methacrylate (GMA), diethylaminoethyl methacrylate (DEAEMA) or a combination thereof. The polymerization initiator may include a thermal initiator or a photoinitiator. Examples of the thermal initiator may comprise, but are not limited to, 2,2′-azobis(2-methylpropionitrile) (AIBN), 2,2′-azodi(2,4-dimethylvaleronitrile) (ADVN), benzoyl peroxide (BPO) or a combination thereof. The photoinitiator may comprise, but are not limited to, 2,4,6-trimethylbenzoyldiphenyl phosphine oxide (TPO), 2-hydroxy-2-methyl propiophenone, 2-methyl-4′-(methylthio)-2-morpholinopropiophenone (Irgacure 907) or a combination thereof. Examples of the cross-linking agent may comprise, but are not limited thereto, ethylene glycol dimethacrylate (EGDMA), triethylene glycol dimethacrylate (TrEGDMA), tetraethylene glycol dimethacrylate (TEGDMA), poly(ethylene glycol) dimethacrylate (PEGDMA), propylene terminal ethylene oxide-dimethylsiloxane-ethylene oxide block copolymer (ABA-type block copolymer), trimethylolpropane trimethacrylate (TMPTMA) or a combination thereof. In one aspect of the present invention, the matrix may further selectively comprise a non-hydrophilic substance. Examples of the non-hydrophilic substance may comprise, but are not limited to, methyl di(trimethylsiloxy) silylpropylglyceryl methacrylate (SIGMMA), 3-(methacryloyloxy)-propyltris(trimethylsiloxy)-silane (TRIS), polydimethylsiloxane (PDMS) or a combination thereof.

Other novel features of the disclosure will become more apparent from the following detailed description when taken in conjunction with the accompanying drawings.

### BRIEF DESCRIPTION OF DRAWINGS

FIG. 1 shows the UV absorption spectra of the UV absorbers according to Examples 1 to 4 and Comparative Examples 1 to 3.

FIG. 2 shows the ultraviolet-visible light transmission spectra of hydrogel films prepared using the UV absorbers of Examples 1 to 4 and Comparative Examples 1 to 3.

### DETAILED DESCRIPTION OF THE INVENTION

The following describes the implementation of the present invention through specific examples. Those skilled in the art can easily understand other advantages and effects of the present invention from the content disclosed in this specification. The present invention can also be implemented or applied through other different specific embodiments. Various details in the present specification can also be modified and changed in various ways according to different viewpoints and applications without departing from the spirit of the present invention.

Unless otherwise stated in the specification, the singular forms “a”, “an” and “the” used in the specification and appended claims include one or the plural individual.

Unless otherwise stated in the specification, the term “or” used in the specification and the appended claims generally includes the meaning of “and/or”.

The present invention will be more specifically illustrated by examples, but these examples are not intended to limit the scope of the present invention. Unless otherwise specified, “%” used to express ingredient content and material mass in the following examples and comparative examples is weight percentage.

### Example 1—Preparation of Compound of Formula (I-1)

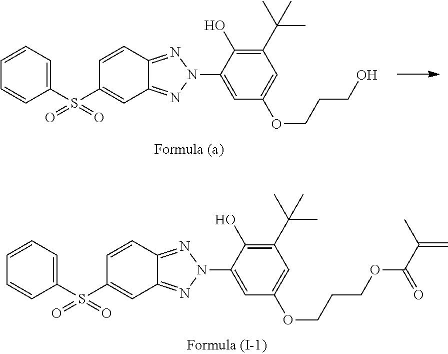 [SMILES: CC(C)(C)c1cc(OCCCO)cc(-n2nc3ccc(S(=O)(=O)c4ccccc4)cc3n2)c1O ; C=C(C)C(=O)OCCCOc1cc(-n2nc3ccc(S(=O)(=O)c4ccccc4)cc3n2)c(O)c(C(C)(C)C)c1]

The compound of formula (a) is 2-(5-benzenesulfonyl-benzotriazol-2-yl)-6-tert-butyl-4-(3-hydroxy-propoxy)-phenol, and the preparation method thereof can be referred to the preparation of formula (I-2) described in TW 1781302.

The compound of formula (a) (120 g), p-toluenesulfonic acid hydrate (14.2 g), hydroquinone (27 mg), methacrylic acid (34.5 g), and toluene (960 g) were added into a reaction bottle. The reaction temperature was raised to 110° C. and refluxed to remove water for 3 hours. After confirming the end point of the reaction with UPLC, the temperature was lowered to normal temperature, and then saturated sodium bicarbonate aqueous solution and toluene were added to perform extraction and separation. The organic layer was concentrated under reduced pressure. The obtained crude product was recrystallized with a methanol-toluene mixture, followed by drying at 80° C. to obtain a yellow solid. The yield was 130 g, and the purity was 97%.

¹H NMR (400 MHz, CDCl₃) δ 1.42 (9H, s), 1.96 (3H, s), 2.22 (2H, qu), 4.15 (2H, t), 4.39 (2H, t), 5.57 (1H, s), 6.13 (1H, s), 7.05 (1H, s), 7.55-7.60 (3H, m), 7.78 (1H, d), 7.90 (1H, d), 8.01-8.03 (3H, m), 8.71 (1H, s), 11.23 (1H, s)

¹³C NMR (100 MHz, CDCl₃) δ 18.5, 28.8, 29.5, 35.9, 61.7, 65.3, 102.9, 118.5, 119.3, 119.8, 125.0, 125.2, 125.7, 128.0, 129.6, 133.7, 136.4, 141.0, 141.1, 141.5, 141.7, 144.0, 144.1, 151.4, 167.5

HRMS (ESI⁺, M+H): calc. 550.2012, found 550.2012

### Example 2—Preparation of Compound of Formula (I-2)

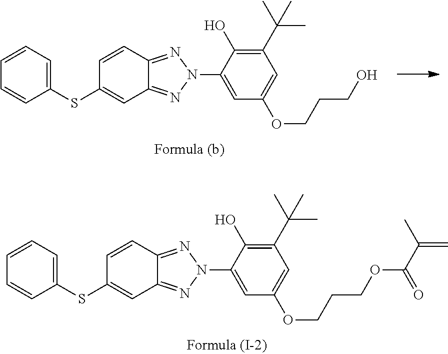 [SMILES: CC(C)(C)c1cc(OCCCO)cc(-n2nc3ccc(Sc4ccccc4)cc3n2)c1O ; C=C(C)C(=O)OCCCOc1cc(-n2nc3ccc(Sc4ccccc4)cc3n2)c(O)c(C(C)(C)C)c1]

The compound of formula (b) is 2-tert-butyl-4-(3-hydroxy-propoxy)-6-(5-phenylsulfanyl-benzotriazol-2-yl)-phenol, and the preparation method thereof can be referred to the preparation of formula (I-1) described in TW 1781302.

The compound of formula (b) (120 g), p-toluenesulfonic acid hydrate (15.2 g), hydroquinone (29 mg), methacrylic acid (34.5 g), and toluene (960 g) were added into a reaction bottle. The reaction temperature was raised to 110° C. and refluxed to remove water for 3 hours. After confirming the end point of the reaction with UPLC, the temperature was lowered to normal temperature, and then saturated sodium bicarbonate aqueous solution and toluene were added to perform extraction and separation. The organic layer was concentrated under reduced pressure. The obtained crude product was recrystallized with a methanol-toluene mixture, followed by drying at 80° C. to obtain a light yellow solid. The yield was 127 g, and the purity was 98%.

¹H NMR (400 MHz, CDCl₃) δ 1.48 (9H, s), 1.95 (3H, s), 2.20 (2H, qu), 4.13 (2H, t), 4.38 (2H, t), 5.56 (1H, s), 6.13 (1H, s), 7.00 (1H, d), 7.36-7.42 (3H, m), 7.48-7.50 (2H, m), 7.67 (1H, d), 7.74 (1H, d), 7.82 (1H, d), 11.43 (1H, s)

¹³C NMR (100 MHz, CDCl₃) δ 18.5, 28.9, 29.5, 35.8, 61.7, 65.2, 102.8, 116.7, 117.3, 118.1, 125.2, 125.7, 128.5, 129.8, 130.0, 132.9, 133.7, 136.4, 137.7, 141.1, 141.7, 143.3, 143.6, 151.2, 167.6

HRMS (ESI⁺, M+H): calc. 518.2114, found 518.2124

### Example 3—Preparation of Compound of Formula (I-3)

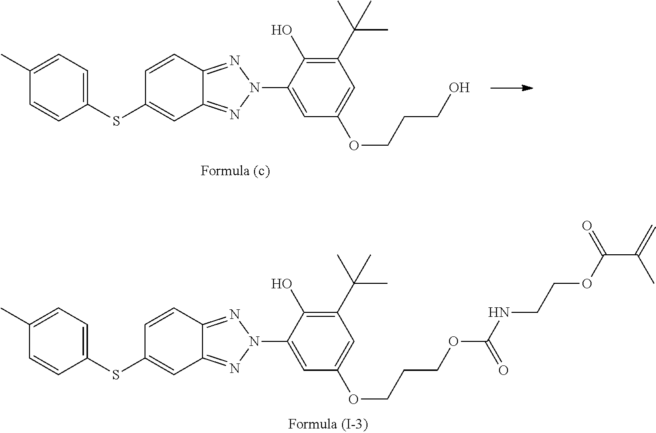 [SMILES: Cc1ccc(Sc2ccc3nn(-c4cc(OCCCO)cc(C(C)(C)C)c4O)nc3c2)cc1 ; C=C(C)C(=O)OCCNC(=O)OCCCOc1cc(-n2nc3ccc(Sc4ccc(C)cc4)cc3n2)c(O)c(C(C)(C)C)c1]

The compound of formula (c) is 2-tert-butyl-4-(3-hydroxy-propoxy)-6-(5-p-tolylsulfanyl-benzotriazol-2-yl)-phenol, and the preparation method thereof can be referred to the preparation of formula (I-1) described in TW I781302.

The compound of formula (c) (29 g), dibutyltin dilaurate (0.26 g) and acetonitrile (150 g) were added into a reaction bottle, followed by heating to 55° C. Isocyanylethyl methacrylate (11.3 g) and acetonitrile (150 g) were slowly added, and the reaction was raised to 60° C. and reacted for 3 hours. After confirming the end point of the reaction with UPLC, the temperature was lowered to normal temperature and a small amount of crystals precipitated. After lowering the temperature to 5-10° C., filtering and drying, a light yellow solid was obtained. The yield was 38 g, and the purity was 97%.

¹H NMR (400 MHz, CDCl₃): δ 1.47 (9H, s), 1.92 (3H, s), 2.11-2.14 (2H, m), 2.40 (3H, s), 3.48-3.52 (2H, m), 4.10-4.11 (2H, t), 4.21-4.24 (2H, t), 4.28-4.31 (2H, t), 5.03 (1H, s), 5.56 (1H, d), 6.10 (1H, d), 6.98-6.99 (1H, d), 7.22-7.26 (2H, m), 7.33-7.34 (1H, d), 7.41-7.44 (2H, m), 7.54 (1H, m), 7.72-7.77 (1H, m), 7.77-7.79 (1H, m), 11.43 (1H, s)

¹³C NMR (400 MHz, CDCl₃): δ 18.4, 21.4, 22.9, 29.2, 29.5, 35.8, 40.3, 51.3, 62.0, 63.8, 65.1, 102.7, 115.2, 117.2, 117.9, 125.2, 126.2, 129.2, 130.6, 133.9, 136.0, 139.0, 139.1, 141.0, 141.5, 143.3, 143.5, 151.2, 156.5, 167.4

HRMS (ESI⁺, M+H): calc. 619.2586, found 619.2586

### Example 4—Preparation of Compound of Formula (I-4)

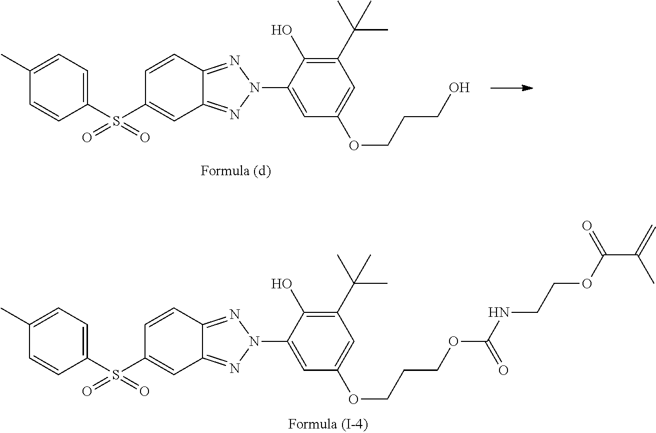 [SMILES: Cc1ccc(S(=O)(=O)c2ccc3nn(-c4cc(OCCCO)cc(C(C)(C)C)c4O)nc3c2)cc1 ; C=C(C)C(=O)OCCNC(=O)OCCCOc1cc(-n2nc3ccc(S(=O)(=O)c4ccc(C)cc4)cc3n2)c(O)c(C(C)(C)C)c1]

The compound of formula (d) is 2-tert-butyl-4-(3-hydroxy-propoxy)-6-[5-(toluene-4-sulfonyl)-benzotriazol-2-yl]-phenol, and the preparation method thereof can be referred to the preparation of formula (I-2) described in TW I781302.

The compound of formula (d) (21.63 g), dibutyltin dilaurate (0.167 g) and acetonitrile (100 g) were added into a reaction bottle, followed by heating to 55° C. Isocyanylethyl methacrylate (7.18 g) and acetonitrile (100 g) were slowly added, and the reaction was raised to 60° C. and reacted for 3 hours. After confirming the end point of the reaction with UPLC, the temperature was lowered to normal temperature and a small amount of crystals precipitated. After lowering the temperature to 5-10° C., filtering and drying, a yellow solid was obtained. The yield was 24 g, and the purity was 96%.

¹H NMR (400 MHz, CDCl₃): δ 1.48 (9H, s), 1.92 (3H, s), 2.11-2.14 (2H, m), 2.40 (3H, s), 3.48-3.52 (2H, m), 4.10-4.13 (2H, t), 4.19-4.24 (2H, t), 4.25-4.33 (2H, t), 5.02 (1H, s), 5.57-5.58 (1H, dd), 6.11 (1H, s), 7.05-7.05 (1H, d), 7.32-7.34 (2H, m), 7.77 (1H, d), 7.87-7.91 (3H, m), 8.00-8.01 (1H, d), 8.68 (1H, s), 11.24 (1H, s)

¹³C NMR (400 MHz, CDCl₃): δ 18.4, 21.7, 29.1, 29.5, 35.8, 40.3, 62.0, 63.8, 65.1, 102.9, 118.4, 119.2, 119.4, 125.0, 125.2, 126.2, 128.1, 130.3, 136.1, 138.1, 141.4, 141.4, 141.7, 144.0, 144.0, 144.8, 151.4, 156.6, 167.4.

HRMS (ESI⁺, M+H): calc. 651.2493, found 651.2493

### Comparative Example 1—RUVA93

Comparative example 1 is a commercially available reactive benzotriazole absorber, RUVA93, and its structure is represented by the following formula (II-1).

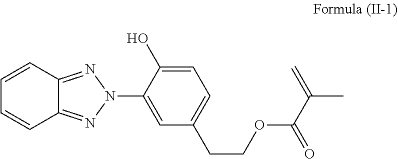 [SMILES: C=C(C)C(=O)OCCc1ccc(O)c(-n2nc3ccccc3n2)c1]

### Comparative Example 2—EV73

Comparative example 2 is a commercially available benzotriazole absorber, EV73, that can absorb the blue light with wavelengths above 380 nm. The structure thereof is represented by the following formula (II-2).

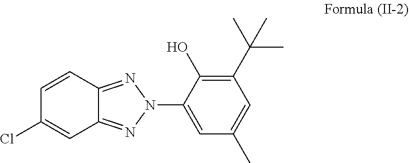 [SMILES: Cc1cc(-n2nc3ccc(Cl)cc3n2)c(O)c(C(C)(C)C)c1]

### Comparative Example 1—Preparation of Compound of Formula (II-3)

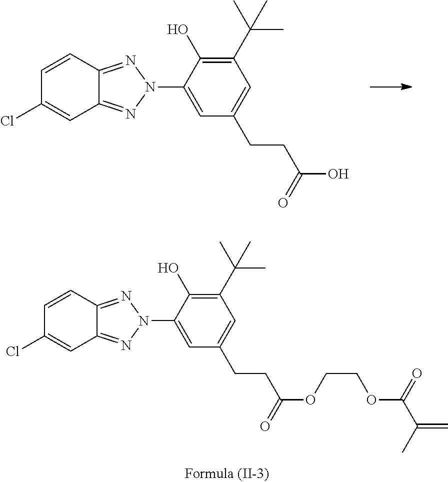 [SMILES: CC(C)(C)c1cc(CCC(=O)O)cc(-n2nc3ccc(Cl)cc3n2)c1O ; C=C(C)C(=O)OCCOC(=O)CCc1cc(-n2nc3ccc(Cl)cc3n2)c(O)c(C(C)(C)C)c1]

Hydroxyethyl methacrylate (15.32 g), 1-ethyl-3-(3-dimethylaminopropyl)carbodiimide (24.8 g) and toluene (100 g) were added into a reaction bottle, followed by introducing nitrogen and stirring. Next, 3-[3-(5-chloro-2H-benzotriazol-2-yl)-5-tert-butyl-4-hydroxyphenyl]propionic acid (40 g) was added, and then toluene (100 g) was added, followed by stirring, heating to 85° C. and reacting for 5 hours. After confirming the end point of the reaction with UPLC, the temperature was lowered to normal temperature. The gummy substance in the lower layer was removed, and the upper layer was extracted with 50% isopropyl alcohol and 1M hydrochloric acid for a few seconds. After removing the water layer, the organic layer was concentrated under reduced pressure and purified by column chromatography (n-hexane:ethyl acetate=8:2). After purification, the liquid was crystallized to obtain a white solid, with a yield of 11.9 g and a purity of 99%.

¹H NMR (400 MHz, CDCl₃): δ 1.49 (9H, s), 1.91 (3H, S), 2.71-2.74 (2H, t), 2.98-3.02 (2H, t), 4.36 (4H, s), 5.55-5.56 (1H, dd), 6.09 (1H, s), 7.21 (1H, d), 7.41-7.43 (1H, m), 7.85-7.91 (2H, m), 8.1 (1H, d), 11.57 (1H, s)

¹³C NMR (400 MHz, CDCl₃): δ 18.4, 29.6, 30.6, 35.6, 36.0, 62.3, 62.5, 116.7, 118.9, 119.0, 125.5, 126.2, 128.4, 129.3, 131.2, 133.6, 136.0, 139.8, 141.3, 141.3, 147.6, 167.2, 172.7

HRMS (ESI⁺, M+H): calc. 486.1797, found 486.1797

### Test Example 1—Comparison of UV Absorption Spectra

The UV absorbers of Examples 1 to 4 and Comparative examples 1 to 3 were respectively formulated into MeOH:THF=9:1 solutions with a concentration of 20 ppm. The absorbance was measured using a UV/visible spectrometer, and the results are shown in Table 1 below. In the present specification, “Example” is abbreviated as “Ex”, and “Comparative example” is abbreviated as “Comp ex”.

TABLE 1

| | | | | | Comp ex | Comp ex | Comp ex |
|---|---|---|---|---|---|---|---|
| | Ex 1 | Ex 2 | Ex 3 | Ex 4 | 1 | 2 | 3 |
| | Formula | Formula | Formula | Formula | Formula | Formula | Formula |
| | (I-1) | (I-2) | (I-3) | (I-4) | (II-1) | (II-2) | (II-3) |
| Molecular | 517.64 | 549.64 | 618.74 | 650.74 | 323.35 | 315.80 | 485.96 |
| weight | | | | | | | |
| (g/mol) | | | | | | | |
| Maximum | 469 | 438 | 437 | 455 | 381 | 407 | 402 |
| absorption | | | | | | | |
| limit | | | | | | | |
| (nm, | | | | | | | |
| A = 0.01) | | | | | | | |

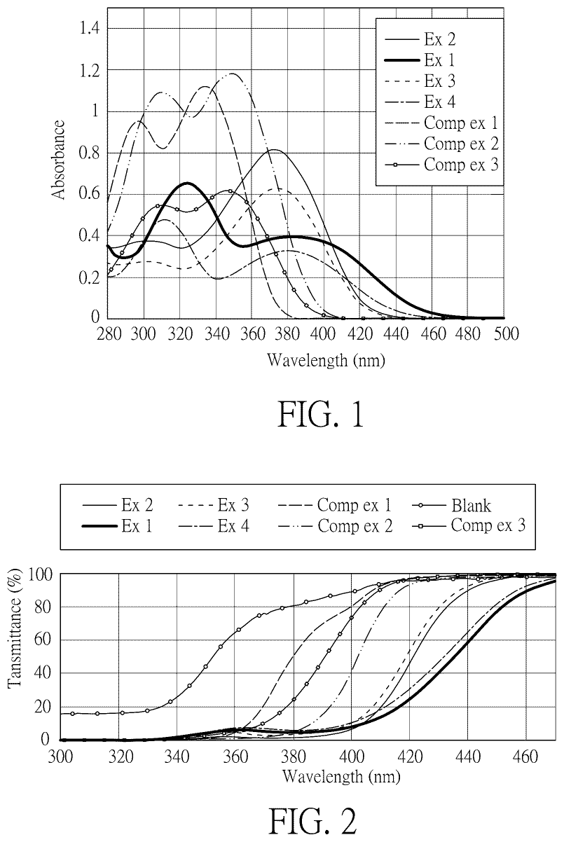

As shown in Table 1 above, compared with Comparative examples 1 to 3, obvious red shifts can be found in the spectra of Examples 1 to 4 (as shown in FIG. 1). The maximum absorption limit (nm, A=0.01) of Examples 1 to 4 is between 437 nm and 469 nm, which is much larger than Comparative example 1 (381 nm) and Comparative examples 2 and 3 (407 nm, 402 nm) with red shift effect. This result indicates that the novel compound provided by the present invention can be used as a reactive UV absorber with excellent anti-blue light effect.

### Test Example 2—Preparation of Hydrogel Film

According to the following table 2, after each component was mixed in proportion, polymerization was carried out by irradiation with a mercury lamp (1500 mJ/cm²), and the hydrogel films with the UV absorbers of Examples 1 to 4 and Comparative Examples 1 to 3 were obtained.

TABLE 2
Formulations of hydrogel films

| | | Ex 1 | Ex 2 | Ex 3 | Ex 4 |
|---|---|---|---|---|---|
| Formulation | | Formula | Formula | Formula | Formula |
| (wt %) | Blank | (I-1) | (I-2) | (I-3) | (I-4) |
| HEMA | 93.95 | 93.70 | 93.70 | 93.70 | 93.70 |
| EGDMA | 1.91 | 1.91 | 1.91 | 1.91 | 1.91 |
| I907 | 0.33 | 0.33 | 0.33 | 0.33 | 0.33 |
| TPO | 1.91 | 1.91 | 1.91 | 1.91 | 1.91 |
| Chemcure 73 | 1.90 | 1.90 | 1.90 | 1.90 | 1.90 |
| UVA | — | 0.25 | 0.25 | 0.25 | 0.25 |
| Total (wt %) | 100.00 | 100.00 | 100.00 | 100.00 | 100.00 |

| | Comp ex 1 | Comp ex 2 | Comp ex 3 |
|---|---|---|---|
| Formulation | Formula | Formula | Formula |
| (wt %) | (II-1) | (II-2) | (II-3) |
| HEMA | 93.70 | 93.70 | 93.70 |
| EGDMA | 1.91 | 1.91 | 1.91 |
| I907 | 0.33 | 0.33 | 0.33 |
| TPO | 1.91 | 1.91 | 1.91 |
| Chemcure 73 | 1.90 | 1.90 | 1.90 |
| UVA | 0.25 | 0.25 | 0.25 |
| Total (wt %) | 100.00 | 100.00 | 100.00 |

HEMA: 2-hydroxyethyl methacrylate
EGDMA: ethylene glycol dimethacrylate
I907: Irgacure 907
TPO: 2,4,6-trimethylbenzoyldiphenyl phosphine oxide
Chemcure 73: 2-hydroxy-2-methyl propiophenone

According to Table 2 above, after mixing the components of the polymerizable mixture in proportion, a hydrogel film was synthesized with a film thickness ranging from approximately 0.18 mm to 0.35 mm. The transmission spectrum of the film can be measured with a UV spectrophotomer. A blank calibration on the set spectrophotometer with a transparent carrier without any added gel or absorbing monomer was performed. Then, the hydrogel films containing the UV absorbers of Examples 1 to 4 and Comparative examples 1 to 3 were placed, and the center positions were confirmed to be at the light source passing position.

The ultraviolet-visible light transmission spectra of the hydrogel films prepared using the UV absorbers of Examples 1 to 4 and Comparative examples 1 to 3 are shown in FIG. 2. The results show that the four novel reactive anti-blue light UV absorbing monomers of the present invention all have excellent blocking effects at 380 nm to 460 nm.

### Analysis of Blue Light Filtering Ability

The average blue light transmittance (%) of the hydrogel films containing the UV absorbers of Examples 1 to 4 and Comparative examples 1 to 3 in the wavelength range of 380-460 nm was measured using a UV-visible light spectrophotometer. The spectral scanning bandwidth is 1 nm, and the blue light blocking rate is used as a reference to evaluate the blue light filtering ability.

A blank calibration on the set spectrophotometer with a transparent carrier without any added gel or absorbing monomer was performed. Next, the hydrogel films containing the UV absorbers of Examples 1 to 4 and Comparative examples 1 to 3 were placed, and the center positions were confirmed to be at the light source passing position. Then, the corresponding blue light filtering ability value was calculated using the following equations (1) and (2). The results are shown in Table 3 below.

### Average Blue Light Transmittance (BLT)

$$BLT \% = \frac{{\sum }_{k = 380 nm}^{460 nm} T ( λ )}{( 460 - 380 ) + 1} \qquad ( 1 )$$

$$Average blue light blocking rate ( BLB )$$

$$BLB \% = 100 \% - BLT \% \qquad ( 2 )$$

TABLE 3

| | | Ex 1 | Ex 2 | Ex 3 | Ex 4 |
|---|---|---|---|---|---|
| | | Formula | Formula | Formula | Formula |
| | Blank | (I-1) | (I-2) | (I-3) | (I-4) |
| Average blue light | 92.7 | 34.2 | 47.9 | 51.7 | 38.7 |
| transmittance | | | | | |
| (%, 380~460 nm) | | | | | |
| Average blue light | 7.3 | 65.8 | 52.1 | 48.3 | 61.3 |
| blocking rate | | | | | |
| (%, 380~460 nm) | | | | | |

| | Comp ex 1 | Comp ex 2 | Comp ex 3 |
|---|---|---|---|
| | Formula | Formula | Formula |
| | (II-1) | (II-2) | (II-3) |
| Average blue light | 88.7 | 71.0 | 83.1 |
| transmittance | | | |
| (%, 380~460 nm) | | | |
| Average blue light | 11.3 | 29.0 | 16.9 |
| blocking rate | | | |
| (%, 380~460 nm) | | | |

As shown in Table 3 above, the hydrogel film still has a slight blue light blocking rate without adding any blue light filtering material (blank), and the reason is that the photoinitiator structure contains aromatic rings. In the case where the UV absorbers of Comparative examples 1 to 3 are added as blue light filtering materials, the results show that the blue light blocking rate of only 11 to 29% can be achieved when adding 0.25 wt % of the UV absorbers. However, when the UV absorbers of Examples 1 to 4 of the present invention are added as blue light filtering materials, the results show that the blue light blocking rate of 48-65% can be achieved when adding 0.25 wt % of the UV absorbers, which is significantly better than Comparative examples 1 to 3.

The above results show that the benzotriazole UV absorber provided by the present invention has both reactive functional groups and anti-blue light function. When the UV absorbers of the present invention and the monomers for contact lens are polymerized to form a film, the average blue light blocking rate can reach more than 48-65%, which is beneficial for use as a blue light filter material for anti-blue light eye devices (such as contact lenses, intraocular lenses, etc.).

The above-mentioned embodiments are only examples for convenience of explanation. The scope of claims of the present invention shall be subject to the scope of the patent application and shall not be limited to the above-mentioned embodiments.

Although the present disclosure has been explained in relation to its embodiment, it is to be understood that many other possible modifications and variations can be made without departing from the spirit and scope of the disclosure as hereinafter claimed.

1. A compound represented by the following formula (I): 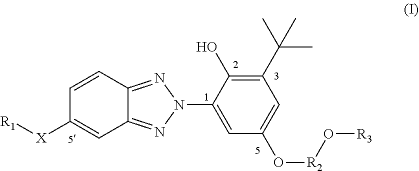
wherein X is —S— or —SO₂—;
R₁ is phenyl or tolyl;
R₂ is linear C₃₋₅ alkylene or a carbamate-containing group; and
R₃ is a C₂₋₅ alkenyl-containing group.

2. The compound of claim 1, wherein R₂ is linear C₃₋₅ alkylene or —R₄—OC(═O)NHR₅—, wherein R₄ is linear C₃₋₅ alkylene and R₅ is linear C₁₋₃ alkylene.

3. The compound of claim 2, wherein R₂ is propylene or —R₄—OC(═O)NHR₅—, wherein R₄ is propylene and R₅ is ethylene.

4. The compound of claim 1, wherein R₃ is a group containing C₂₋₅ alkenyl and carbonyl.

5. The compound of claim 4, wherein R₃ is —C(═O)—R₆ and R₆ is C₂₋₅ alkenyl.

6. The compound of claim 5, wherein R₆ is propenyl.

7. The compound of claim 1, which is any compound of the following formulas (I-1) to (I-4):

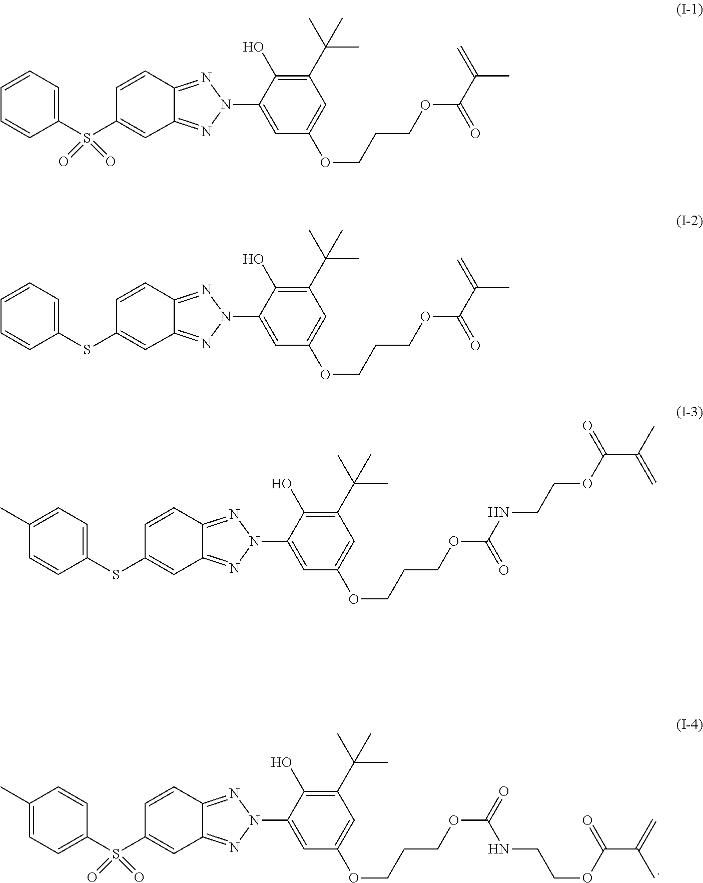

8. An anti-blue light eye device with anti-blue light effect, comprising a compound represented by the following formula (I): 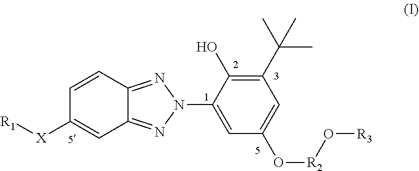
wherein X is —S— or —SO₂—;
R₁ is phenyl or tolyl;
R₂ is linear C₃₋₅ alkylene or a carbamate-containing group; and
R₃ is a C₂₋₅ alkenyl-containing group.

9. The anti-blue light eye device of claim 8, wherein the anti-blue light eye device is a contact lens or an intraocular lens.

10. The anti-blue light eye device of claim 8, wherein R₂ is linear C₃₋₅ alkylene or —R₄—OC(═O)NHR₅—, wherein R₄ is linear C₃₋₅ alkylene and R₅ is linear C₁₋₃ alkylene.

11. The anti-blue light eye device of claim 9, wherein R₂ is propylene or —R₄—OC(═O)NHR₅—, wherein R₄ is propylene and R₅ is ethylene.

12. The anti-blue light eye device of claim 8, wherein R₃ is a group containing C₂₋₅ alkenyl and carbonyl.

13. The anti-blue light eye device of claim 12, wherein R₃ is —C(═O)—R₆ and R₆ is C₂₋₅ alkenyl.

14. The anti-blue light eye device of claim 13, wherein R₆ is propenyl.

15. The anti-blue light eye device of claim 8, wherein the compound is any compound of the following formulas (I-1) to (I-4): [chemical structure]
